(19) United States
(12) Patent Application Publication
(10) Pub. No.: US 2018/0177456 A1
(43) Pub. Date: Jun. 28, 2018

(54) APPARATUS AND METHOD FOR DETECTING INFECTIONS

(71) Applicants: Elsa Swanson, Philadelphia, PA (US); Forrest Miller, Philadelphia, PA (US); Emily Cen, Littlel Neck, NY (US); David Xing, Providence, RI (US)

(72) Inventors: Elsa Swanson, Philadelphia, PA (US); Forrest Miller, Philadelphia, PA (US); Emily Cen, Littlel Neck, NY (US); David Xing, Providence, RI (US)

(21) Appl. No.: 14/956,813

(22) Filed: Dec. 27, 2016

(51) Int. Cl. A61B 5/00; G01N 33/96; A61B 5/07; A61B 5/145; A61B 5/1459; A61B 5/1495; A61F 2/38

(52) U.S. Cl. CPC A61B 5/4851; G01N 33/96; A61B 5/076; A61B 5/14546; A61B 5/1459; A61B 5/1495; A61F 2002/3067; A61B 5/746; A61F 2/389; G01N 2496/05; A61B 2562/0233; A61B 2562/164; A61B 5/686

(57) ABSTRACT

This invention relates to diagnostic medical instruments and procedures, and more particularly to implantable devices and methods for monitoring physiological parameters. A device for providing in vivo diagnostics of infections in orthopedic implants having at least one signal processing device operatively coupled with sensors. The signal processing device is operable to receive the output signal from the sensors and transmit a signal corresponding with the output signal. The invention also relates to a method using the device of the invention for detecting infection associated with implants in a human or animal subject.

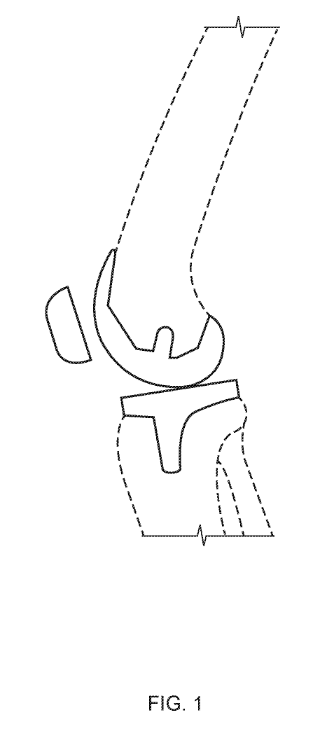

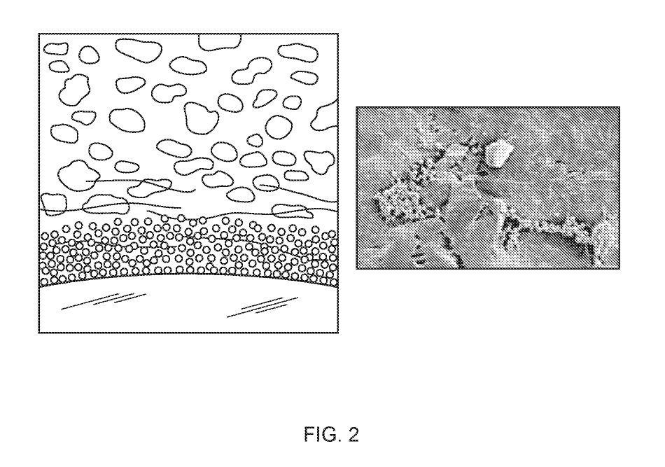

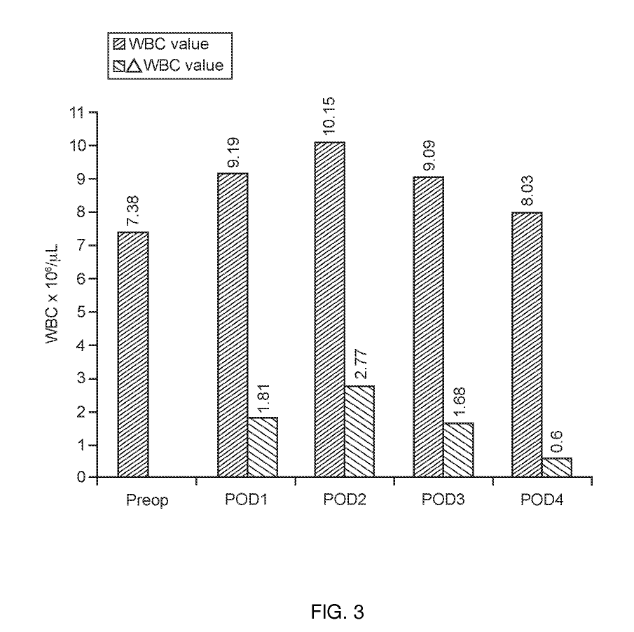

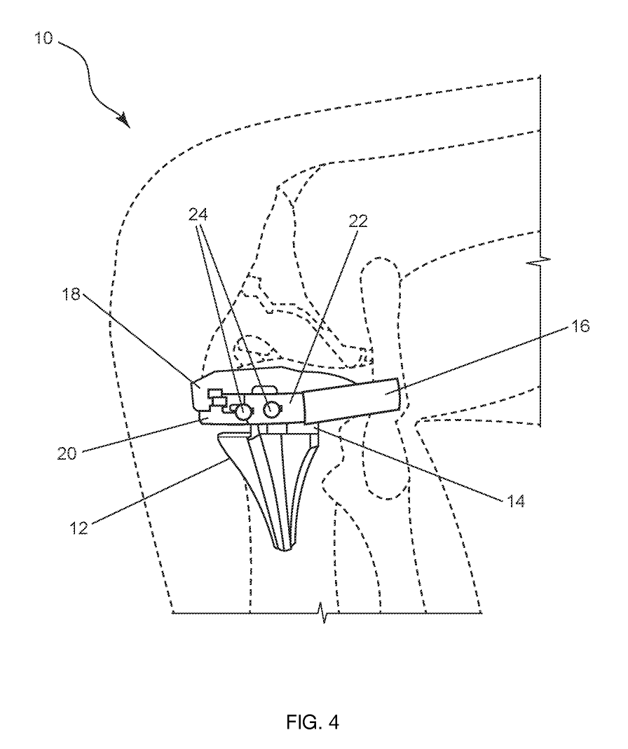

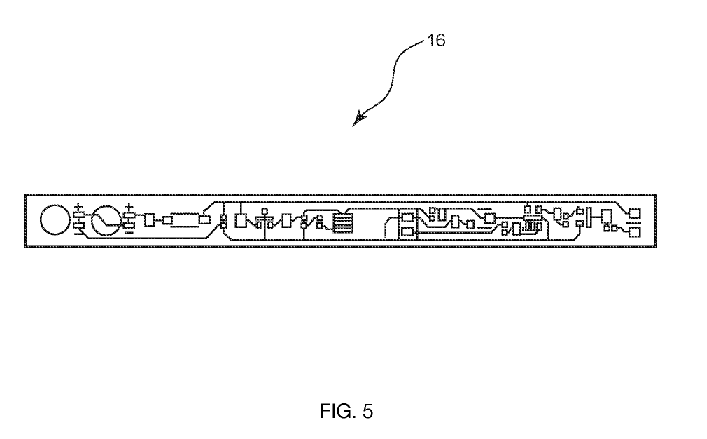

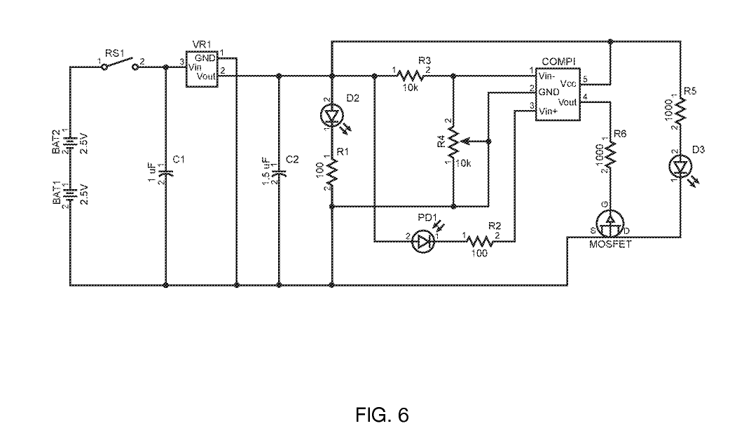

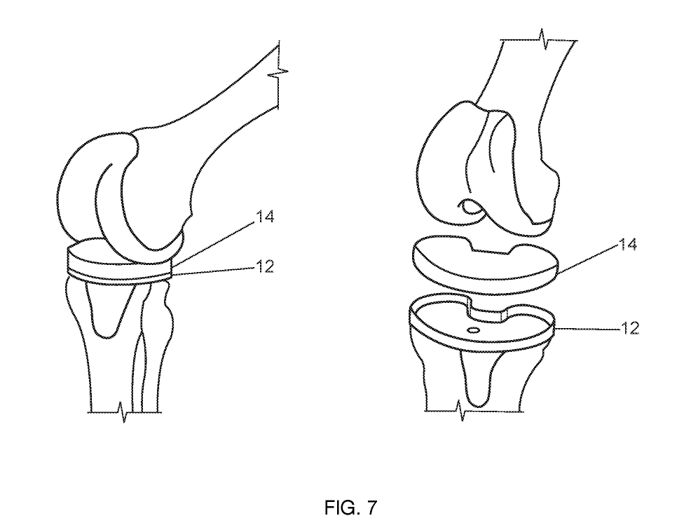

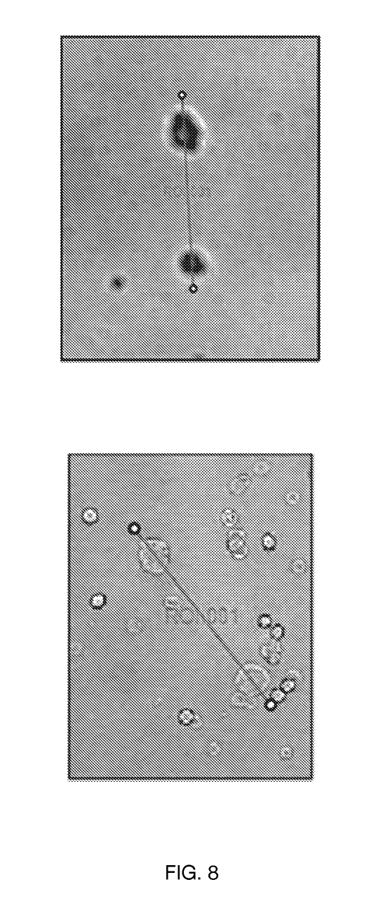

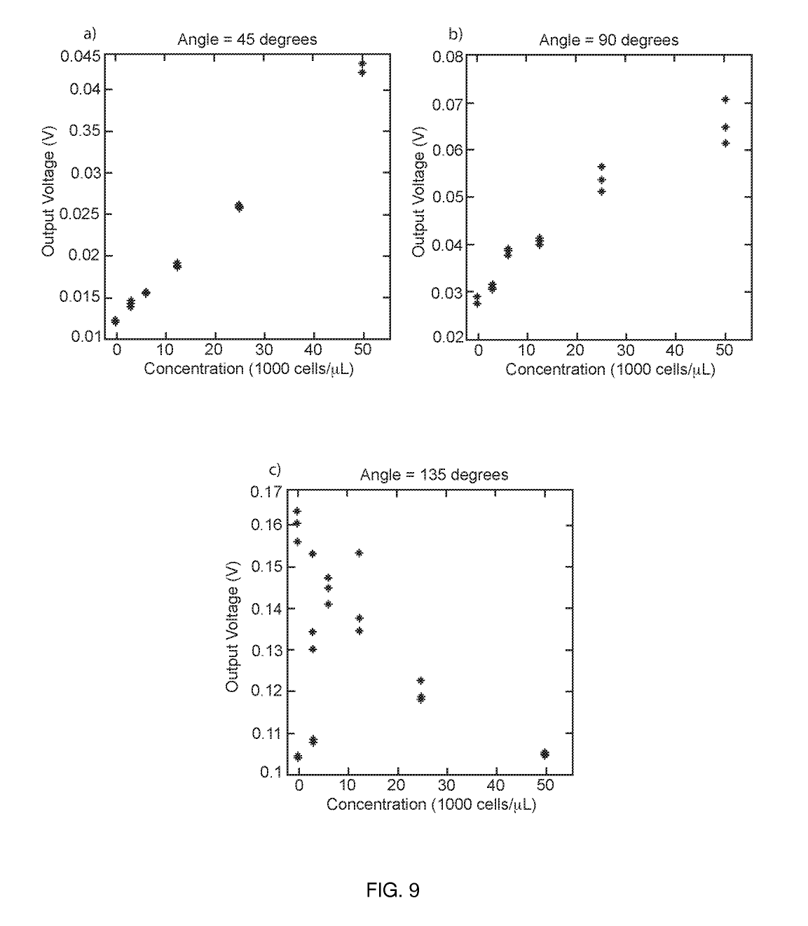

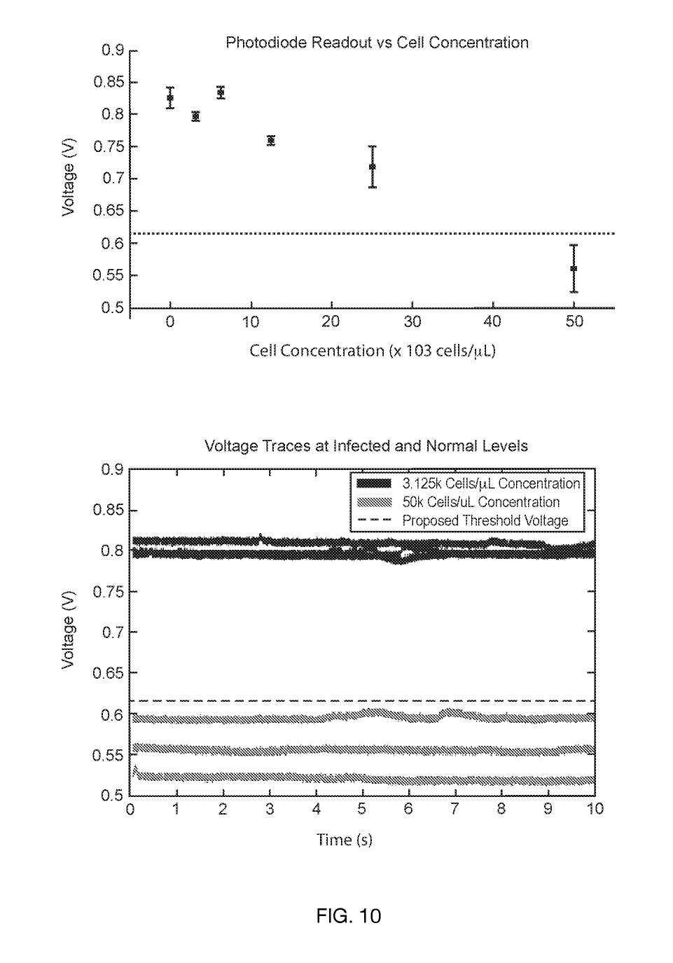

### CROSS-REFERENCE TO RELATED APPLICATIONS

This application claims the benefit under 35 USC § 119(e) of U.S. Provisional Application No. 62/060,533 filed Oct. 6, 2014, and the entire content is incorporated by reference herein and made part of this specification.

### BACKGROUND

The present invention provides an apparatus for and a method of detecting infection in implanted artificial joints in a human or animal subject employing an implanted LED light and sensor integrated directly into the implant. The present invention provides an improved apparatus and method to the currently used methods, which includes physicians relying on indirect measurements such as blood tests, self-report of pain, and observation of inflammation at the site of implantation, all of which can prove inconclusive until an infection is well-advanced. The apparatus of the present invention is scalable to meet a variety of infection detection needs in a variety of joints.

Total knee arthroplasty is one of the most commonly performed procedures in the United States, and periprosthetic infection is the primary cause of premature implant failure. Most postoperative infections can be treated with antibiotics if detected early, but many go undiagnosed until it is too late to save the implant. One million two hundred thousand (1,200,000) prosthetic joints are implanted annually in the United States. Approximately twenty-four thousand (24,000) infections are projected to occur as a result of implantation surgery each year, for a rate of ˜two percent (2%). Implanted medical devices in general cause up to one million infections each year. Suppression of infection around implanted prostheses can require up to six weeks or more of antibiotic treatment, significantly decreasing a patient's quality of life. Severe infections require highly invasive revision surgeries at an average cost of $30,000.00 (US) per surgery.

Total knee arthroplasty surgery was introduced over forty years ago. There have been numerous advances in the procedure and incremental advances in materials used since that time. Currently, plastic used in the spacer, cement used to hold the implant in place, and metal used in the device all continue to be optimized. These incremental improvements in the materials of the implant were virtually the only innovations in implant design until recently.

In May of 2004, the first so-called “smart knee” was implanted in a patient. This device was able to measure compressive forces in vivo, quantifying the implants ability to bear the load of the patient, which is its primary biomechanical function. Two years later, a new knee was developed that could record and wirelessly report data from twelve different strain gauges. This data could be analyzed and converted into a three-dimensional map of every force on the knee, including not only compression force but rotational and shear stresses as well. The entire system was powered without batteries with an external electric field used to induce charge in a coil. The coil was integrated with the sensing device proximal to the implant.

Other prospective technologies designed to ensure the best possible patient outcomes, including various incarnations of instrumented knees, include a device that senses the presence of bacteria and acts to disrupt them via the administration of antibiotics and an electric field, and a trial polyethylene liner equipped with an array of strain gauges.

Periprosthetic infection, defined as when a pathogen is isolated by culture from at least two separate tissue or fluid samples obtained from the affected prosthetic joint, is the most common cause of failure of implantable medical devices. Infections at or near artificial joints require costly and highly invasive revision surgeries, which can risk further infection, scarring, as well as permanent damage to the affected tissues. Many implant patients are treated with prophylactic administration of antibiotics, but approximately 2% of the 1,200,000 artificial joints implanted annually still become infected. Infection due to an implanted device is relatively difficult to detect in early stages of the infection. Physicians currently rely on indirect measurements such as blood tests, self-report of pain, and observation of inflammation at the site of implantation, all of which can prove inconclusive until an infection is well-advanced. Periprosthetic infections occur at a rate of approximately 24,000 per year in the United States and are the most common cause of implant failure. Serious infection around the site of implanted devices can require expensive and dangerous revision surgery.

A reliable, integrated method of detecting and reporting periprosthetic infection of implanted knees or other joints in both humans and animals in vivo while the implant can still be saved is needed. Customers include hospitals, orthopedics companies, patients, and doctors. Stakeholders include hospitals, physicians and nurses, orthopedics companies, patients, Medicare and private insurance companies.

The technology that the apparatus and method of the current invention employs directly detects bacteria existing at the site of implantation and comprises a wireless apparatus that alerts both patient and physician. The benefits of using the technology comprising the present invention allow physicians to detect periprosthetic infections early when treatment options are more numerous, and also to avoid costly and highly invasive revision surgeries.

The present invention provides an apparatus for and a method of detecting infection in implanted artificial joints comprising an implanted LED light and sensor integrated directly into the implant. The present invention detects infection at very early stages when treatment options are much less invasive by monitoring the synovial fluid circulating around the artificial joint, thus decreasing the need for costly and dangerous revision surgeries.

The apparatus and method of the present invention overcomes the deficiencies of the devices and methods currently used because the method is straightforward and elegant and results in significantly improved performance. The apparatus and method of the present invention more fully meets functional requirements because it detects the presence of infection prior to biofilm formation, i.e. no later than two weeks post-infection, and it notifies the patient and/or physician if infection is detected.

The apparatus and method of the present invention is an improvement on currently available devices and methods because it is biocompatible, doesn't interfere with mechanical properties or functionality of an implant, wear and stress tests provide same results with and without the apparatus of the present invention integrated on implant, and it costs significantly less than revision surgery. The present invention works for at least one month post-surgery, is simple for a surgeon to implement, does not interfere with current surgical process, is small enough not to interfere with human body processes, does not cause discomfort or injury to patient, there is no requirement of additional surgery, and is of high specificity and high sensitivity (>80% sensitivity and >70% specificity).

In summary, the present invention provides for an improved method and apparatus for detecting infections near implants.

### SUMMARY

The present invention comprises an apparatus for detecting infection comprising: a flexible circuit board comprising an optical detector comprising an implanted LED and a photodiode, wherein said flexible circuit board is adhered to a surface of an implant wherein said surface comprises a titanium tibial component of an implant. The present invention further comprises an apparatus that is activated wirelessly by a magnet or other signal comprising an RF.

The present invention further comprises an apparatus comprising a photosensor device sensitive to distinguish between high and low WBC counts, which are sufficiently correlated to the turbidity of synovial fluid and further detects infection prior to biofilm formation with a high specificity and sensitivity.

The present invention further comprises an apparatus comprising elements which provide an output voltage reading wirelessly and comprise biocompatible elements. The present invention further comprises an apparatus comprising a recess on the scale of a few millimeters that is disposed in a non-articulated surface of a prosthetic joint; an LED light source; and a photosensor disposed in said recess, thus providing access to synovial fluid of a joint without compromising prosthetic function.

The present invention further comprises a conducting coil in a biocompatible sheath disposed distal or proximal to an implant, which when subject to an external alternating electric field supplies an induced current to the device; and a data transmission system capable of communicating complex.

The present invention further comprises a method of providing a smartphone application that interprets and displays data, as well as an apparatus.

Furthermore, the method of use of the present invention comprises a procedure wherein a surgeon extracts synovial fluid and thus obtains a preoperative baseline white blood cell concentration used to calibrate the present invention, which is on the scale of microns. The method of use of the present invention further comprises the steps of illustrating output voltage vs white blood cell concentration data obtained in final testing of the apparatus and implementing the optimal 90° configuration of the elements of the apparatus.

The method of use of the present invention further comprises providing improvements that explicate expected false positive rates of the sensor in vivo, discovering compounds in synovial fluid other than white blood cells and bacterial cells; and triggering an alarm after providing for ideal placement of sensors; directly detecting bacteria existing at the site of implantation; comprising a wireless apparatus that alerts both patient and physician; and providing high specificity and high sensitivity, wherein providing high specificity and high sensitivity comprises providing greater than 80% sensitivity and greater than 70% specificity.

The method of use of the present invention further comprises disposing a photodiode and an LED and an RF transmitter adjacent to the implant; tripping an RF transmitter when a detector increases above a threshold level; and detecting transmitted light by an external apparatus, such as a smartphone.

The method of use of the present invention further comprises detecting infection comprising the following the steps of creating the polyethylene component of an implant comprising creating an indentation into a lateral surface of that component; placing an optical detector and photodiode in the indentation at a 90° orientation with the appropriate circuitry; and disposing an implant into a patient.

The method of use of the present invention further comprises detecting infection comprising the steps of creating a temporary spacer; creating an indentation into a lateral surface of the spacer; placing an optical detector and photodiode in the indentation at a 90° orientation with the appropriate circuitry; and disposing an implant as part of a two-stage exchange revision procedure.

The method of use of the present invention further comprises creating an arbitrary implantable medical device which is perfused by fluid in vivo; creating an indentation into a nonarticulating surface of that device; placing an optical detector and photodiode in the indentation at a 90° orientation with circuitry; and disposing the implant into the patient.

### DETAILED DESCRIPTION

The present invention addresses and improves on deficiencies in devices currently used. The technology of the present invention comprises a recess on the scale of a few millimeters that is disposed in a non-articulated surface of a prosthetic joint, an LED light source, and a photosensor placed in the recess, providing access to the synovial fluid of the joint without compromising prosthetic function. The photosensor is precisely calibrated to detect very small changes in the quality of the fluid flowing through the joint. Buildup of white blood cells due specifically to the presence of bacterial cells at high concentrations is detected easily by the photosensor. Changes in quality above a certain level trigger a wireless notification device, alerting both patient and physician to the presence of infection at the site of implantation. Early detection and treatment of periprosthetic infection allows for better patient outcomes after implant surgery.

Applications of the present invention include detection of infection in artificial joints, detection of infection in arterial stents, detection of infection in ventricular shunts, detection of infection in fracture-fixation devices, detection of infection in implanted pacemaker-defibrillators, and with modifications to provide for embodiments of the present invention, detection and simultaneous suppression of infection in implanted medical devices.

The present invention provides improvements that explicate expected false positive rates of the sensor in vivo, discover compounds in synovial fluid other than white blood cells and bacterial cells, and triggers an alarm. The present invention provides for ideal placement of sensors. The present invention provides for an effective infection center and prevents sensor clogging in a body. The present invention provides for an effective power source for LED, detector, and transmitter.

The preferred embodiment of the present invention comprises a conducting coil in a biocompatible sheath either distal or proximal to the implant, which when subject to an external alternating electric field supplies an induced current to the device. The preferred embodiment of the present invention comprises a data transmission system capable of communicating complex, i.e. >1 bit, data. An alternate embodiment comprises a battery rather than the described inductive coupling powering system and a data transmission system that will only communicate one bit of data.

The knee is a joint in the human body, and is located where the thigh joins the lower leg. It consists of the lower part of the femur, the upper part of the tibia, and the patella or kneecap. There is a layer of cartilage surrounding the area where the bones meet to prevent bone wear and allow a smooth range of motion for the joint. There are menisci between the tibia and femur as well, which absorb shock in the joint. All the surfaces are covered by the synovial membrane, which is a thin layer of tissue that secretes lubricant which maintains the low friction within the joint. There are four major ligaments in the knee: the anterior and posterior cruciate ligaments and the medial and lateral collateral ligaments. When the cartilage wears away, the bones may start rubbing against each other, causing damage to the surface of the tibia and femur. In such cases, a knee arthroplasty is conducted, where the surfaces of the bones are replaced with a biocompatible metal and polyethylene prosthesis which allows full movement in the sagittal plane.

Infection may occur during and after the arthroplasty surgery. Table 1 illustrates the most common microbes associated with periprosthetic joint infection. Table 1 shows microbiological results in prosthetic knee Infections diagnosed at hospital clinic of Barcelona (Spain) from 2007 to 2009.

TABLE 1

| Microorganism | % Infection |
|---|---|
| S. aureus | 55.8% |
| ECN | 17.6% |
| E. faecalis | 8.8% |
| E. coli | 8.8% |
| E. cloacae | 2.9% |
| K. pneumoniae | 2.9% |
| P. aeruginosa | 2.9% |

Microbes may obtain access to the prosthesis either during surgery through contamination of the wound, or from hematogenous seeding or spread from a contiguous infection. Chronic infection often results in loosening of the implant at the bone-cement interface. Infection can be treated with a strong course of antibiotics, usually vancomycin or linezolid, if the infection is detected early enough. A number of tests can be performed in an effort to detect and diagnose a periprosthetic infection, but they all have associated drawbacks and there is currently no one preferred method.

A few examples of currently used methods include radionucleotide imaging where exact techniques vary. PET (positron emission tomography) imaging of indium-111 leukocytes is the best method currently used, but the specificity and sensitivity are variable; serology where elevated levels of serum C-reactive protein (CRP) and Erythrocyte Sedimentation Rate (ESR)—both measures of inflammation—are tested for. The test is performed at three months post-surgery, long after a biofilm may have formed; cultures which exhibit high sensitivity and specificity, but only if performed within two weeks following antibiotic discontinuation. Gram stains have low specificity and sensitivity, meaning the test has trouble distinguishing the staph and strep bacteria that are likely to form biofilms. Other examples of currently used methods include frozen sections of implant membranes which are similar in outcomes and limitations of joint fluid leukocyte counts. Newer tests include tests for bacterial rRNA that have been investigated and found to be susceptible to false positives, as they are sensitive to dead bacteria as well as living ones; joint fluid leukocyte counts where this method is sensitive and specific, and forms the scientific basis of the present invention, but standard tests are invasive, inconvenient and slow. Another newer test is for the proinflammatory cytokine IL-6, for which there exists specialized labs. This testing is highly accurate, but extremely time-consuming as it must be performed remotely.

The drawbacks to methods currently used include that after the bacteria have colonized the surface of the prosthetic, a biofilm begins to form. This biofilm is a major characteristic of periprosthetic joint infection. Studies have shown that surface characteristics of the prosthetic contribute to increased biofilm formation, including increased roughness, hydrophobicity, and the lack or presence of any antimicrobial coatings. Furthermore, proteins such as fibronectin promote adherence of bacteria to biomaterial surfaces. Cell surface polymers are involved in attachment to hydrophobic surfaces while lipopolysaccharides are more involved in attachment to hydrophilic surfaces. The bacteria synthesize extracellular polymeric substances (EPS), which are resistant to antibiotics and the host's immune system, resulting in the culture surviving and subsequent infection. These EPSs are primarily composed of polysaccharides, and are highly hydrated. Thus, in general, an infection at the site of the implant must be detected and treated before the bacteria are able to form a biofilm, because a device on which a biofilm has formed will continue to serve as a literal breeding ground for bacteria until the biofilm is removed. Low-energy surface acoustic waves have been shown to disrupt biofilm formation in Foley catheters and increase the bacteria's vulnerability to antibiotics but this has not yet been replicated in orthopedic implants.

Normal synovial fluid white blood cell count after surgery with no presence of infection: White blood cell count is a useful test for infection shortly post-operatively because, in contrast to other measurements such as CRP and ESR, white blood cell count returns to preoperative levels within several days to a week post-surgery. If there is an infection, the concentration rises by up to two orders of magnitude.

Periprosthetic infections (PPIs) in total knee arthroplasties are defined as meeting one or more of the following criteria: an abscess or sinus tract communicating with the joint space, a positive preoperative culture of aspirate, at least two positive intraoperative cultures of the same organism, or a positive culture in addition to either gross intracapsular purulence or abnormal histological findings.

These infections are classified into three types based on the timing of the infection. These categories are acute postoperative (up to four weeks post-surgery), late chronic (over four weeks postoperative), and hematogenous (acute onset at a previously well-functioning prosthetic joint). Acute postoperative (Stage I) infections are due to bacteria gaining access to the joint during or soon after the operation from either the skin or a draining wound. These infections usually show symptoms within a few days or weeks post-surgery. Late chronic (Stage II) infections result from bacteria from air, surgical instruments, or the implant itself. The delay before symptoms present is due to the time organisms need to proliferate before becoming symptomatic.

Lastly, in hematogenous (Stage III) infections, the organism is carried to the arthroplasty site via the bloodstream. In this scenario, a prior infection can enter the implant site. Alternatively, an acute surgical site infection can become systemic if the surgical site infection enters the bloodstream.

Of the four classifications of periprosthetic infections, acute and hematogenous infections are more likely to be caused by more virulent pathogens such as Staphylococcus bacteria. When total knee replacements become infected, two of the three most predominant infectious organisms are strains of Staphylococcus bacteria: Staphylococcus aureus and Staphylococcus epidermidis. The clinical presentations of Staph. vary depending on the location and severity of the infection. Boils, impetigo, cellulitis, and pus may develop on the skin. Blood poisoning can occur if the bacteria enter the bloodstream. If the patient develops toxic shock syndrome because of the Staph. infection, the associated symptoms are fever, nausea, vomiting, rash, confusion, seizures, headaches, and muscle aches.

In the more specific case of knee replacement infections, however, pain is the predominant clinical symptom and other symptoms are frequently absent or overlap with symptoms of other implant complications such as aseptic loosening. If other symptoms do present themselves, they can be in the form of swelling, erythema, local warmth, or drainage post-surgery.

The present invention takes advantage of previous research and comprises an optical detection device that comprises and LED and a photodiode incorporated onto the lateral tibial surface of the implant. Apparatus of the present invention and its method of use and placement in vivo: A polyethylene component is disposed adjacent to the tibial plate. Elements are inserted into the user's tibia which supports the tibial plate. Batteries supply power to a flex circuit.

There are at present methods of testing for ESR, CRP, and IL-6 post TKA surgery. The bacteria enter the arthroplasty site. Symptoms may present within days or weeks post-surgery. The white blood cell count, ESR and CRP levels in the blood of the patient are elevated as a result of the body's natural reaction to major surgery. However, these results can also be seen if the patient has chronic inflammatory rheumatic disease; as such it is imperative to establish a preoperative baseline for any measurements that will later be used for diagnostic purposes. CRP levels return to normal two to three weeks post-surgery and, ESR may take 6 weeks or more to return to normal levels if an infection is not present. However, they will stay elevated if there is an infection. White blood cell count within the synovial fluid, however, returns to normal levels as early as four days post-surgery if an infection has not begun.

A circuit schematic of the present invention: Two 2.5V batteries power the apparatus of the present invention, which is activated by the reed switch (RS1). The voltage regulator (VR1) sets a constant 2.8V to the rest of the circuit to eliminate any unwanted fluctuations. The photodiode output is fed into 1 lead of the comparator (COMP1) and a set threshold voltage, controlled by the potentiometer (R4) is fed into the other lead of the comparator. The comparator sends a high voltage to its V-out lead, connected to the gate of a MOSFET, when the photodiode voltage crosses the threshold. The MOSFET closers the infrared LED circuit, lighting it up, when the gate is high. This IR signal is transmitted outside of the knee to an external sensing device.

The placement of the apparatus of the present invention in the implant is crucial to obtaining the most accurate measurement, as well as maintaining the integrity of the implant itself. The present invention apparatus is positioned on the lateral surface of the implant. The lateral surfaces of the tibial component or the polyethylene spacer of the implant are the preferred locations for measuring infection. The sides of the implant surfaces are non-articulating surfaces, which are directly adjacent to the synovial sacks. This orientation prevents the mechanically crucial articulating surfaces from being compromised, as well as disposes the present invention in a location where it is readily perfused by fluid from the constantly regenerating synovial sacks.

The WBC count in the synovial fluid increases sharply, often by orders of magnitude, if a bacterial infection has started to form. Specifically, the WBC count will increase from approximately 4,200 cells/μL to 92,600 cells/μL, or higher. Most of the WBCs that flood the synovial fluid in response to infection are neutrophils, which change the color of the synovial fluid, turning it from transparent to an opaque white-yellow. The elevated response persists throughout infection, though once a biofilm forms, as discussed below, the body is essentially powerless to eradicate the bacteria.

Within the first few weeks after surgery, a biofilm may form if the bacteria arrive at the implant prior to the arrival of benevolent human proteins and growth factors. Biofilms form approximately two weeks after bacteria have arrived at the site of the implant. One week later, human leukocytes attach to the biofilms, but fail to phagocytose biofilm bacteria. Immediately post-surgery, the synovial fluid is perfused with red blood cells and has a similar composition to that of blood. However, a human body actively tries to return the composition of the synovial fluid back to normal by producing more synovial fluid via the synovial sacs. The red blood cell content is completely resorbed from the joint between two to five months post-surgery.

The clinical outcomes of Staphylococcus infections include death, irreversible damage to organ systems (such as endocarditis, pneumonia and osteomyelitis), and sepsis if the bacteria enter the human bloodstream. Septic arthritis may also develop, and if left untreated, the joint may be destroyed and the infection may spread to other parts of the body.

Before an infection can be treated, it must be diagnosed. The doctor first has to suspect infection before any tests are run to confirm the clinician's suspicion. These diagnostic tests include various imaging techniques (such as radiographs, radionuclide bone scans, and PET scans), testing for biomarkers such as WBC count, ESR, and CRP levels in blood tests, cultures, and assays. Once the infection has been diagnosed, patients who are deemed unable to undergo a revision arthroplasty (the risk of surgery outweighs the benefits of replacement) are given a six-week program of intravenous antibiotics. These patients will have extremely poor quality of life, as the antibiotics alone will not cure the infection at this stage. Alternatively, an operative debridement can be performed and the infected prosthesis retained. This type of treatment is only suitable for acute postoperative PPIs and hematogenous PPIs that have been identified early. A last alternative to a full revision is a resection arthroplasty. This procedure is reserved for patients that cannot undergo the more extensive full revision surgery as its functional results are poor compared to that of full revisions.

There are two types of full revision surgeries: single-stage exchange and two-stage exchange revisions. The single-stage exchange revision surgery consists of the use of an antibiotic-loaded cement along with surgical debridement and a postoperative 6-week (minimum) course of parenteral antibiotics. This technique works best for patients with an acute infection (stages I and III). Otherwise, a two-stage exchange revision is generally preferred. In this revision surgery, the first stage consists of removing all infected tissues and hardware and inserting an antibiotic-loaded spacer. The present invention is deployed on the lateral surface of this spacer to monitor joint health throughout the exchange process. A six (6)-week (minimum) course of parenteral antibiotics is administered also at this time. Finally, when appropriate, the temporary spacer is removed and the new prosthesis is implanted.

However, many patients do not present with clinical symptoms, meaning the infection will not be diagnosed until it is too late and a revision arthroplasty must be performed. Other times, even if the patient presents with symptoms, the infection is caught too late; by the time the test results to confirm the diagnosis return, a revision surgery is required. The present invention addresses and solves this and other problems. Total joint arthroplasty (TJA) is a cost-effective procedure with high rates of success in alleviating pain and improving knee function in patients with advanced arthritis of the knee. There has been a steady rise in the number of revision surgeries performed post-primary TJA in the United States. This increase is due to a number of factors, including an increase in the number of primary TJA procedures (with the growing aging population), modifications to surgical procedures, implant life-expectancy, and the expansion of the patient population to include younger, more active patients.

Though white blood cell (WBC) count does increase in the synovial fluid post-operatively, it will return to preoperative levels within 4 days after the surgery. Therefore, WBC count can be used as an early detection method. White blood cell count in the synovial fluid increases from 4,200 cells/μL to 92,600 cells/μL on average in the case of infection. Therefore, WBC count in synovial fluid is used as an accurate predictor of infection, with a high sensitivity of 84% and specificity of 99%. Furthermore, research has shown that the concentration of WBCs, particularly neutrophils, is causative of and highly correlated with the optical turbidity of the synovial fluid.

A millimeter scale indentation is cut into the titanium surface such that synovial fluid diffuses freely into the space containing the detector and the LED, which are oriented at 90° with respect to one another. The photodiode registers voltage based on the amount of light scattered by the cells present in the space. White blood cells infiltrate the synovial space and cause a quality change in the fluid when there is presence of an infection. Light is both absorbed and scattered by the white blood cells as the fluid entering into the space becomes saturated with white blood cells. The detector trips an LED transmitter once the voltage in the detector increases past a threshold value, which then notifies the patient and/or physician of the infection.

Optimizing LED and photodiode orientation: This parameter is crucial, as it determines the mechanical embodiment of the device circuit, and therefore of any alterations made to the prosthesis itself, as well as the relative contributions of scattering and absorption characteristics to the total increase or attenuation of the output voltage.

Output voltage vs white blood cell concentration data obtained in final testing of the device in its preferred embodiment: Having implemented the optimal 90° configuration, final testing in a physically accurate phantom knee test bed was performed with the present embodiment of the invention.

The preferred embodiment of the present invention is incorporated into the titanium tibial component of the implant. An alternate embodiment of the present invention will be disposed in the surface of the polyethylene component.

The present invention detects infection prior to biofilm formation with a high specificity and sensitivity because WBC count in synovial fluid has been shown to satisfy the important conditions previously discussed as being critical for early infection detection. The present invention provides an output voltage reading wirelessly, which satisfies the notification of diagnosis requirement. All components of the present invention are biocompatible, and therefore easily integrated into both the implant and the knee environment. Furthermore, since it is integrated into the implant, the device is not difficult for the surgeon to implement.

During the procedure the surgeon is able to extract synovial fluid and obtain a preoperative baseline white blood cell concentration used to calibrate the present invention. The present invention is on the scale of microns, so it is unlikely to cause injury or discomfort to the patient.

The present invention solves the following challenges: what is the relation between light scattering/absorption and white blood cell count? Which wavelength gives the best correlation between WBC count and light scattering/absorption? What is the sensitivity and range of our photosensor, and is it good enough for infection detection?

The present invention provides an apparatus comprising a photosensor device sensitive enough to distinguish between high and low WBC counts, which are sufficiently correlated to the turbidity of the synovial fluid, and thereby detects when the immune system is reacting to an infection.

The present invention comprises an apparatus that determines the set of parameters that result in the best correlation between WBC count and light scattering/absorption and determines what form of wireless communication is most effective in transmitting a signal through at least 1.5 inches of tissue.

### EXAMPLE 1

### An Optical Comparison of WBCs and MSCs Was Made

Optical analysis of mesenchymal stem cells (MSC) was performed using a confocal microscope. A sample of MSCs were pipetted onto a glass slide and imaged using the confocal microscope. Images were taken 50 μm above the focal plane to measure absorbance profiles of the cells, and at eight different wavelengths, namely 458, 476, 488, 496, 514, 543, 594, and 633 nm. In order to make an optical comparison, a small sample of diluted whole blood was placed on a glass slide, and images were taken with the same settings as listed before.

Once images were obtained using the confocal microscope, analysis of these images was performed using the Leica Viewing software to make a comparison of the absorbance profiles for WBCs and MSCs. Regions of interest were selected along a straight line across two cells, and the intensity of signal across that region of interest was recorded.

### EXAMPLE 2

### Spectrophotometer Analysis of MSCs in Synovial Fluid

A sample of 0.735E6 cells/mL of MSCs in PBS were obtained and separated using a centrifuge set to 4.5 for 7 minutes. The MSCs were carefully removed by pouring off the top layer of liquid, and the cells were resuspended in 120 μL of bovine synovial fluid in a test tube, resulting in a concentration of 50,000 cells/μL. This new solution of MSCs in bovine synovial fluid was extensively mixed using a pipette in order to ensure uniform concentration of cells in the test tube. A 96 well plate was used to allow measurement for small samples of fluid since the number of cells available for testing was limited. In the initial well, 60 μL of the MSCs in synovial fluid solution was carefully inserted to ensure the entire bottom of the well was covered. In order to perform analysis of varying concentrations of MSCs in synovial fluid, another 60 μL of synovial fluid was added to the test tube, and again mixed thoroughly. In the second well, 60 μL of the diluted solution (25,000 cells/μL) was inserted, and the same dilution process was performed for four more wells.

The spectrophotometer analysis was performed on six wells in a 96 well plate with varying concentrations of MSCs in synovial fluid, namely: 50,000 cells/μL, 25,000 cells/μL, 12,500 cells/μL, 6,250 cells/μL, 3,125 cells/μL, and o cells/μL. A full spectral sweep was performed to analyze the best wavelength to observe differences in cell concentration.

### EXAMPLE 3

### Initial Circuit With LED and Photodiode

A preliminary circuit containing an LED from the lab in series with a 1.71 kΩ resistor and powered by a PSU supplying 5V was built. On the other side of the circuit, the photoresistor (Thorlabs FDS100) was added, with a bias voltage supplied by the PSU at 5V. The photoresistor was connected to an RC filter to remove noise (R=100Ω, C=1 μF), and also connected in series to a load resistor (1 MΩ) in order to measure output current. The LED was positioned so that the light beam would face the photoresistor surface, as shown below.

### EXAMPLE 4

### Optical Comparison, WBCs and MSCs and Spectrophotometer Analysis of MSCs in Synovial Fluid

Regions of interest were selected, and resulting absorbance profiles measured for the two cells in both the MSC and WBC sample. Regions of interest were selected across a. two MSCs, and b. across two WBCs. The smaller more round cells in b. are red blood cells.

### EXAMPLE 5

### Optimizing LED and Photodiode Orientation

Results of testing the output voltage vs. concentration at three angles: 45°, 90°, and 135°: There is a positive correlation between voltage and cell concentration at 45° and 90° and a negative correlation between the two at 135°.

Results indicated that the circuit and components were sensitive enough to distinguish between infected levels and normal physiological levels of WBC count at 45° and 90° conformations. Therefore, either orientation is used in the present invention. The 90° orientation is the preferred embodiment because of its higher voltage output and because this conformation provided a greater degree of freedom when incorporating the present invention onto an implant. This conclusion was confirmed in our final test bed. There is a very clear distinction between infected and physiologically normal WBC count levels.

Output voltage vs. concentration results for final testing using a latest test bed. The final LED and photodiode parts were oriented at 90° and 5 mm from each other. The results show an ˜250 mV difference between infected and physiological levels, indicating that a clear distinction between the two cases could be made based on photodiode voltage alone. The voltage difference is negative because of this final embodiment's reverse voltage bias, which was implemented due to battery considerations.

Although the invention has been described in detail with particular reference to these preferred embodiments, other embodiments can achieve the same results. Variations and modifications of the present invention are obvious to those skilled in the art and it is intended to cover all such modifications and equivalents.

We claim:

1. The apparatus for detecting infection comprising:
a flexible circuit board comprising an optical detector comprising an implanted LED and a photodiode, wherein said flexible circuit board is adhered to a surface of an implant wherein said surface comprises a titanium tibial component of an implant.

2. The apparatus of claim 1 wherein said apparatus is activated wirelessly by a magnet or other signal comprising an RF.

3. The apparatus of claim 1 further comprising a photosensor device sensitive to distinguish between high and low WBC counts, which are sufficiently correlated to the turbidity of synovial fluid.

4. The apparatus of claim 1 that further detects infection prior to biofilm formation with a high specificity and sensitivity.

5. The apparatus of the present invention of claim 1 further comprising elements which provides an output voltage reading wirelessly.

6. The apparatus of the present invention of claim 1 further comprising biocompatible elements.

7. The apparatus of claim 1 further comprising:
a recess on the scale of a few millimeters that is disposed in a non-articulated surface of a prosthetic joint;
an LED light source; and a photosensor disposed in said recess, thus providing access to synovial fluid of a joint without compromising prosthetic function.

8. The method of use of the present invention comprising a procedure wherein a surgeon extracts synovial fluid and obtains a preoperative baseline white blood cell concentration used to calibrate the present invention, which is on the scale of microns.

9. The method of use of the present invention of claim 8 further comprising;
illustrating output voltage vs white blood cell concentration data obtained in final testing of the apparatus; and
implementing the optimal 90° configuration of the elements of the apparatus.

10. The method of use of the present invention of claim 8 further comprising:
providing improvements that explicate expected false positive rates of the sensor in vivo, discovering compounds in synovial fluid other than white blood cells and bacterial cells; and
triggering an alarm after providing for ideal placement of sensors;
directly detecting bacteria existing at the site of implantation;
comprising a wireless apparatus that alerts both patient and physician; and
providing high specificity and high sensitivity.

11. The method of use of claim 8 wherein providing high specificity and high sensitivity comprises providing greater than 80% sensitivity and greater than 70% specificity.

12. The apparatus of claim 1 further comprising:
a conducting coil in a biocompatible sheath disposed distal or proximal to an implant, which when subject to an external alternating electric field supplies an induced current to the device; and
a data transmission system capable of communicating complex.

13. The apparatus of claim 1 further comprising:
providing a smartphone application that interprets and displays data.

14. The method of claim 8 further comprising:
disposing a photodiode and an LED and an RF transmitter adjacent to the implant;
tripping the RF transmitter when a detector increases above a threshold level;
detecting transmitted light by an external apparatus, such as a smartphone.

15. The method of claim 8 further comprising:
detecting infection comprising the steps of creating the polyethylene component of an implant:
creating an indentation into a lateral surface of that component; and
placing an optical detector and photodiode in the indentation at a 90° orientation with the appropriate circuitry; and disposing an implant into a patient.

16. The method of claim 8 further comprising:
detecting infection comprising the steps of:
creating a temporary spacer;
creating an indentation into a lateral surface of the spacer;
placing an optical detector and photodiode in the indentation at a 90° orientation with the appropriate circuitry; and
disposing an implant as part of a two-stage exchange revision procedure.

17. The method of claim 8 for detecting infection further comprising:
creating an arbitrary implantable medical device which is perfused by fluid in vivo;
creating an indentation into a nonarticulating surface of that device;
placing an optical detector and photodiode in the indentation at a 90° orientation with circuitry; and
disposing the implant into the patient.
